SEQUENCE LISTING (70 sequences)

SEQ ID NO: 1 - protein, 5 aa, Arabidopsis sp.
SSSMR

SEQ ID NO: 2 - protein, 5 aa, Arabidopsis sp.
SSSLR

SEQ ID NO: 3 - protein, 22 aa, Arabidopsis sp.
DQQQNHDSSSMRSTRLQRFLSL

SEQ ID NO: 4 - protein, 17 aa, Arabidopsis sp.
DQQQNHDSTRLQRFLSL

SEQ ID NO: 5 - DNA, 20 bp, Arabidopsis sp.
  source 1..20: mol_type = unassigned DNA
atcccgatttctccggtcca

SEQ ID NO: 6 - DNA, 23 bp, Arabidopsis sp.
  source 1..23: mol_type = unassigned DNA
actttctccttgcgatgggaaga

SEQ ID NO: 7 - DNA, 24 bp, Arabidopsis sp.
  source 1..24: mol_type = unassigned DNA
aagattcgatctctctcatcgagt

SEQ ID NO: 8 - DNA, 18 bp, Arabidopsis sp.
  source 1..18: mol_type = unassigned DNA
ggagtggtccgttgagca

SEQ ID NO: 9 - DNA, 20 bp, Arabidopsis sp.
  source 1..20: mol_type = unassigned DNA
cgacctttcaaccgtcttat

SEQ ID NO: 10 - DNA, 21 bp, Arabidopsis sp.
  source 1..21: mol_type = unassigned DNA
tcgagaagctaaaccagagaa

SEQ ID NO: 11 - DNA, 20 bp, Arabidopsis sp.
  source 1..20: mol_type = unassigned DNA
tcagcactttccagcagatg

SEQ ID NO: 12 - DNA, 19 bp, Arabidopsis sp.
  source 1..19: mol_type = unassigned DNA
ctgtggacaatgcctggac

SEQ ID NO: 13 - DNA, 57 bp, Arabidopsis sp.
  source 1..57: mol_type = unassigned DNA
gactctagaaagcttctgcaggggcccgggatgaagcaactctggactttcttcttc

SEQ ID NO: 14 - DNA, 51 bp, Arabidopsis sp.
  source 1..51: mol_type = unassigned DNA
tcatggtctttgtagtccatggtaccgtctaatggatttaccgaattgaag

SEQ ID NO: 15 - DNA, 32 bp, Arabidopsis sp.
  source 1..32: mol_type = unassigned DNA
acgcgtcgacatggcggatcagctaaccgatg

SEQ ID NO: 16 - DNA, 34 bp, Arabidopsis sp.
  source 1..34: mol_type = unassigned DNA
ctagactagttcactttgccatcatgactttgac

SEQ ID NO: 17 - DNA, 43 bp, Arabidopsis sp.
  source 1..43: mol_type = unassigned DNA
ccccaattgatggcggatcagctcacagacgatcagatctcag

SEQ ID NO: 18 - DNA, 43 bp, Arabidopsis sp.
  source 1..43: mol_type = unassigned DNA
ccgctcgagtcacttagccatcataaccttcacaaactcttcg

SEQ ID NO: 19 - DNA, 42 bp, Arabidopsis sp.
  source 1..42: mol_type = unassigned DNA
ccggaattcatggcggatcagctaaccgatgaccagatctcc

SEQ ID NO: 20 - DNA, 42 bp, Arabidopsis sp.
  source 1..42: mol_type = unassigned DNA
ccgctcgagtcactttgccatcatgactttgacgaactcttc

SEQ ID NO: 21 - DNA, 36 bp, Arabidopsis sp.
  source 1..36: mol_type = unassigned DNA
acgcgtcgaccttatgtgggctgtcacgggttgttg

SEQ ID NO: 22 - DNA, 45 bp, Arabidopsis sp.
  source 1..45: mol_type = unassigned DNA
ccaattgttaatggtgatggtgatgatggtctaatggatttaccg

SEQ ID NO: 23 - DNA, 35 bp, Arabidopsis sp.
  source 1..35: mol_type = unassigned DNA
acgcgtcgacctcgctctcttcctatactttgttc

SEQ ID NO: 24 - DNA, 45 bp, Arabidopsis sp.
  source 1..45: mol_type = unassigned DNA
ggaattcttaatggtgatggtgatgatggtctaatggatttaccg

SEQ ID NO: 25 - DNA, 31 bp, Arabidopsis sp.
  source 1..31: mol_type = unassigned DNA
acgcgtcgacaaaataatccgcgacttctgc

SEQ ID NO: 26 - DNA, 44 bp, Arabidopsis sp.
  source 1..44: mol_type = unassigned DNA
ccggaattcttaatggtgatggtgatgatgtatgggtcttctag

SEQ ID NO: 27 - DNA, 34 bp, Arabidopsis sp.
  source 1..34: mol_type = unassigned DNA
acgcgtcgacttgatgatacgtcagtttggacag

SEQ ID NO: 28 - DNA, 45 bp, Arabidopsis sp.
  source 1..45: mol_type = unassigned DNA
ccggaattcttaatggtgatggtgatgatggttgcagcgacttga

SEQ ID NO: 29 - DNA, 35 bp, Arabidopsis sp.
  source 1..35: mol_type = unassigned DNA
acgcgtcgaccagatcatccgtcagctctacaatg

SEQ ID NO: 30 - DNA, 45 bp, Arabidopsis sp.
  source 1..45: mol_type = unassigned DNA
ccggaattcttaatggtgatggtgatgatgatctaatggattaac

SEQ ID NO: 31 - DNA, 34 bp, Arabidopsis sp.
  source 1..34: mol_type = unassigned DNA
acgcgtcgaccacgcctgcaacctcttctaccag

SEQ ID NO: 32 - DNA, 45 bp, Arabidopsis sp.
  source 1..45: mol_type = unassigned DNA
ccggaattcttaatggtgatggtgatgatggcatgaagcggtggt

SEQ ID NO: 33 - DNA, 42 bp, Arabidopsis sp.
  source 1..42: mol_type = unassigned DNA
aggctagcctcgaggaattcatgggttctcatcatcatcatc

SEQ ID NO: 34 - DNA, 44 bp, Arabidopsis sp.
  source 1..44: mol_type = unassigned DNA
atgagtttttgttcggatcctcacttcgctgtcatcatttgtac

SEQ ID NO: 35 - DNA, 50 bp, Arabidopsis sp.
  source 1..50: mol_type = unassigned DNA
caggtgtcgtgaacacgtggtcgcggccgcatgttaagctcaatgaattg

SEQ ID NO: 36 - DNA, 50 bp, Arabidopsis sp.
  source 1..50: mol_type = unassigned DNA
gagatctggcctcgagtccggtaccgaattcatatgggtcttctagatgc

SEQ ID NO: 37 - DNA, 49 bp, Arabidopsis sp.
  source 1..49: mol_type = unassigned DNA
caggtgtcgtgaacacgtggtcgcggccgcatgaagcaactctggactt

SEQ ID NO: 38 - DNA, 49 bp, Arabidopsis sp.
  source 1..49: mol_type = unassigned DNA
gagatctggcctcgagtccggtaccgaattcagtctaatggatttaccg

SEQ ID NO: 39 - DNA, 48 bp, Arabidopsis sp.
  source 1..48: mol_type = unassigned DNA
caggtgtcgtgaacacgtggtcgcggccgcatgaagtggtttctgctt

SEQ ID NO: 40 - DNA, 49 bp, Arabidopsis sp.
  source 1..49: mol_type = unassigned DNA
gagatctggcctcgagtccggtaccgaatttagttgcagcgacttgaac

SEQ ID NO: 41 - DNA, 48 bp, Arabidopsis sp.
  source 1..48: mol_type = unassigned DNA
caggtgtcgtgaacacgtggtcgcggccgcatgaagctactctggtct

SEQ ID NO: 42 - DNA, 52 bp, Arabidopsis sp.
  source 1..52: mol_type = unassigned DNA
gagatctggcctcgagtccggtaccgaattcaatctaatggattaaccgaat

SEQ ID NO: 43 - DNA, 49 bp, Arabidopsis sp.
  source 1..49: mol_type = unassigned DNA
caggtgtcgtgaacacgtggtcgcggccgcatgaagttcattttctatc

SEQ ID NO: 44 - DNA, 48 bp, Arabidopsis sp.
  source 1..48: mol_type = unassigned DNA
gagatctggcctcgagtccggtaccgaatttagcatgaagcggtggtg

SEQ ID NO: 45 - DNA, 52 bp, Arabidopsis sp.
  source 1..52: mol_type = unassigned DNA
caggtgtcgtgaacacgtggtcgcggccgcatggcggatcagctcacagacg

SEQ ID NO: 46 - DNA, 48 bp, Arabidopsis sp.
  source 1..48: mol_type = unassigned DNA
gagatctggcctcgagtccggtaccgaattcacttagccatcataacc

SEQ ID NO: 47 - DNA, 52 bp, Arabidopsis sp.
  source 1..52: mol_type = unassigned DNA
caggtgtcgtgaacacgtggtcgcggccgcatggcggatcagctaaccgatg

SEQ ID NO: 48 - DNA, 50 bp, Arabidopsis sp.
  source 1..50: mol_type = unassigned DNA
gagatctggcctcgagtccggtaccgaattcactttgccatcatgacttt

SEQ ID NO: 49 - DNA, 25 bp, Arabidopsis sp.
  source 1..25: mol_type = unassigned DNA
gattgcttgcaatggcatcatcggt

SEQ ID NO: 50 - DNA, 25 bp, Arabidopsis sp.
  source 1..25: mol_type = unassigned DNA
aaacaccgatgatgccattgcaagc

SEQ ID NO: 51 - DNA, 25 bp, Arabidopsis sp.
  source 1..25: mol_type = unassigned DNA
gattgatctcttgcaatggcatcat

SEQ ID NO: 52 - DNA, 25 bp, Arabidopsis sp.
  source 1..25: mol_type = unassigned DNA
aaacatgatgccattgcaagagatc

SEQ ID NO: 53 - DNA, 25 bp, Arabidopsis sp.
  source 1..25: mol_type = unassigned DNA
gattgttgcaaacgagtggagcgca

SEQ ID NO: 54 - DNA, 25 bp, Arabidopsis sp.
  source 1..25: mol_type = unassigned DNA
aaactgcgctccactcgtttgcaac

SEQ ID NO: 55 - DNA, 25 bp, Arabidopsis sp.
  source 1..25: mol_type = unassigned DNA
gattgcaagaatctttgcaaacgag

SEQ ID NO: 56 - DNA, 25 bp, Arabidopsis sp.
  source 1..25: mol_type = unassigned DNA
aaacctcgtttgcaaagattcttgc

SEQ ID NO: 57 - DNA, 41 bp, Arabidopsis sp.
  source 1..41: mol_type = unassigned DNA
ttgcaagaatggatgagaaagaagagtccaagcacgaaagc

SEQ ID NO: 58 - DNA, 43 bp, Arabidopsis sp.
  source 1..43: mol_type = unassigned DNA
catccattcttgcaatggcatcatcggttggctttttatagag

SEQ ID NO: 59 - DNA, 43 bp, Arabidopsis sp.
  source 1..43: mol_type = unassigned DNA
catgactccactcgtttgcaaagattcttgtctctcatggatg

SEQ ID NO: 60 - DNA, 42 bp, Arabidopsis sp.
  source 1..42: mol_type = unassigned DNA
acgagtggagtcatgattctgctgttgatctcttgcaatggc

SEQ ID NO: 61 - DNA, 19 bp, Arabidopsis sp.
  source 1..19: mol_type = unassigned DNA
attttgccgatttcggaac

SEQ ID NO: 62 - DNA, 21 bp, Arabidopsis sp.
  source 1..21: mol_type = unassigned DNA
gatgctgcatatggttgtgtg

SEQ ID NO: 63 - DNA, 21 bp, Arabidopsis sp.
  source 1..21: mol_type = unassigned DNA
gttgaacgataagcttgcgag

SEQ ID NO: 64 - DNA, 21 bp, Arabidopsis sp.
  source 1..21: mol_type = unassigned DNA
ttcgttcaaaggtggcataac

SEQ ID NO: 65 - DNA, 21 bp, Arabidopsis sp.
  source 1..21: mol_type = unassigned DNA
cgactatgaggaaagacgcag

SEQ ID NO: 66 - protein, 84 aa, Arabidopsis sp.
QIIRQLYKKPTDDAIARDQQQNHDSSSMRSTRLQRFLSLMDEKEESKHESKKRKIDGSMNDTSGSTRSRGFDRERSFNSVNPLD

SEQ ID NO: 67 - protein, 54 aa, Arabidopsis sp.
QIIRQLYKKPLQEINSRIMTLPPCAPLVCKDSCLSWMRKKSPSTKARRERSMVQ

SEQ ID NO: 68 - protein, 23 aa, Arabidopsis sp.
QIIRQLYKKPTDECHCKRSTAES

SEQ ID NO: 69 - protein, 79 aa, Arabidopsis sp.
QIIRQLYKKPTDDAIARDQQQNHDSTRLQRFLSLMDEKEESKHESKKRKIDGSMNDTSGSTRSRGFDRERSFNSVNPLD

SEQ ID NO: 70 - protein, 41 aa, Arabidopsis sp.
QIIRQLYKKPTDDAIARDQQQNHDSSSMRSTRFAKILVSHG

(19) United States
(12) Patent Application Publication
(10) Pub. No.: US 2025/0270584 A1
(43) Pub. Date: Aug. 28, 2025

(54) Gene editing to provide insect resistance in crops

(71) Applicant: The Regents of the University of California, Oakland, CA (US)

(72) Inventors: Sheng Luan, Berkeley, CA (US); Chun Yan, Berkeley, CA (US)

(73) Assignee: The Regents of the University of California, Oakland, CA (US)

(21) Appl. No.: 19/200,689

(22) Filed: May 7, 2025

Related U.S. Application Data
(63) Continuation of application No. PCT/US23/81045, filed on Nov. 25, 2023.
(60) Provisional application No. 63/431,238, filed on Dec. 8, 2022.

(51) Int. Cl. C12N 15/82; C07K 14/415

(52) U.S. Cl. CPC C12N 15/8286; C07K 14/415

(57) ABSTRACT

Methods and compositions to produce insect resistance in plants deploy an engineered glutamate receptor-like protein (GLR3.3) that functions in transmitting wound-induced signals from local to systemic plant organs to trigger synthesis of jasmonic acid, a defense hormone.

### CROSS-REFERENCES TO RELATED APPLICATIONS

This application is a continuation of PCT/US23/81045, filed Nov. 25, 2023, which claims priority to U.S. Provisional Application No. 63/431,238, filed Dec. 8, 2022, the disclosures of which are hereby incorporated by reference in its entirety for all purposes.

### GOVERNMENT SUPPORT CLAUSE

This invention was made with government support under GM138401 awarded by the National Institutes of Health. The government has certain rights in the invention.

### REFERENCE TO A SEQUENCE LISTING

A Sequence Listing in XML format is incorporated by reference into the specification. The name of the XML file containing the Sequence Listing is B22-085-2US.xml. The XML file is 63,862 bytes and was created on May 7, 2025.

### INTRODUCTION

Plants rely on systemic signaling mechanisms to establish whole-plant defense in response to insect and nematode attack. The Glutamate receptor-like (GLR) genes have been implicated in long-distance propagation of wound signals to initiate accumulation of defense hormone jasmonate (JA) at undamaged distal sites. The systemic signaling entails activation of Ca²⁺-permeable GLR channels by wound-released glutamate (Glu), triggering membrane depolarization and cytosolic Ca²⁺ influx throughout the plants. The systemic electrical signals and calcium waves rapidly dissipate to restore the resting state.

### SUMMARY OF THE INVENTION

We disclose calmodulin (CaM)-mediated desensitization of GLR channels in a Ca²⁺ dependent manner, revealing a negative feedback loop to orchestrate plant systemic wound responses. Engineered GLR3.3 alleles with impaired desensitization provide prolonged systemic electrical signaling and Ca²⁺ waves, leading to enhanced plant defense against herbivores. Moreover, this Ca²⁺/CaM-mediated desensitization of GLR channels is a highly conserved mechanism in plants, providing a target for engineering anti-herbivore defense in crops.

The invention provides methods and compositions to produce insect resistance in plants, especially crops, deploying an engineered glutamate receptor-like protein (GLR3.3) that functions in transmitting wound-induced signals from local to systemic plant organs to trigger synthesis of jasmonic acid, a defense hormone. The GLR3.3 is a glutamate-gated, calcium permeable channel that is inhibited by calmodulin (CaM). We identified the CaM-binding domain of GLR3.3 and made mutations in this domain using CRISPR procedure, defining engineered mutants that provide a hypersensitive response to wounding, resulting in enhanced resistance to insect feeding. The mutant plants are otherwise normal and show same productivity.

The invention provides aspect and embodiments:

A method of gene editing to provide insect resistance in a plant, the method comprising engineering Ca²⁺/calmodulin-mediated desensitization of a glutamate receptor to enhance plant systemic wound signaling and anti-herbivore defense.

A method of attenuating or impairing Ca²⁺/CaM-mediated GLR3.3 channel desensitization compared with wild type GLR3.3, without affecting Glu-activated channel opening, comprising expressing in the plant an engineered GLR protein herein.

A method of increasing expression of JA-responsive genes or elevates wound-induced accumulation of jasmonic acid (JA) or its bioactive derivative JA-Ile in systemic leaves of the plant, as compared to wild type plants, comprising expressing in the plant an engineered GLR protein herein.

A method of prolonging or enhancing durations of wound- and/or Glu-triggered slow wave potentials (SWPs) compared to wild type, or increased systemic SWPs and Ca²⁺ waves owing to impaired Ca²⁺/CaM-mediated channel desensitization, comprising expressing in the plant an engineered GLR protein herein.

A method of enhancing plant systemic wound signaling and anti-herbivore defense, comprising expressing in the plant an engineered GLR protein herein.

An engineered glutamate receptor-like (GLR) protein, GLR3.3, comprising a gain-of-function mutation in the calmodulin (CaM)-binding domain (CBD) within cytoplasmic C-terminal domain (CTD) corresponding to residues 867-888 of Arabidopsis wild type GLR3.3, wherein the protein provides in a plant:
attenuated or impaired Ca²⁺/CaM-mediated GLR3.3 channel desensitization compared with wild type GLR3.3, without affecting Glu-activated channel opening; or
increased expression of JA-responsive genes or elevated wound-induced accumulation of jasmonic acid (JA) or its bioactive derivative JA-Ile in systemic leaves of the plant, as compared to wild type plants; or
prolonged or enhanced durations of wound- and/or Glu-triggered slow wave potentials (SWPs) compared to wild type, or increased systemic SWPs and Ca²⁺ waves owing to impaired Ca²⁺/CaM-mediated channel desensitization.

A protein of any claim wherein the GLR3.3 if from Cruciferae species Arabidopsis, Brassica napus, Brassica oleracea, or Raphanus sativus.

A protein of any claim wherein mutant comprises a deletion of amino acid residues within the CBD.

A protein of any claim wherein the mutant comprises a deletion of 1, 2, 3, 4 or 5 residues of a target amino acid sequence SSSMR (SEQ ID NO:1) or SSSLR (SEQ ID NO:2).

A recombinant polynucleotide encoding an engineered protein herein.

A cell comprising a recombinant polynucleotide herein, and expressing the engineered GLR3.3 protein.

A plant comprising a cell herein, comprising a recombinant polynucleotide herein, and expressing the engineered GLR3.3 protein.

The invention encompasses all combinations of the particular embodiments recited herein, as if each combination had been laboriously recited.

### BRIEF DESCRIPTION OF THE DRAWING

FIG. 1. GLR3.3 CTD plays a dual role in propagating systemic electrical signaling. Schematic subunit structure of GLR3.3 channel with the approximate locations of T-DNA insertion mutation (glr3.3a) and CRISPR-engineered mutations (T1˜T4) marked by filled triangles. ATD, amino-terminal domain; LBD, ligand-binding domain; M1˜M3, membrane spanning segments.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Example: Engineering Ca²⁺/Calmodulin-Mediated Desensitization of Glutamate Receptors to Enhance Plant Systemic Wound Signaling and Anti-Herbivore Defense

Despite lack of a classical nerve system, plants have evolved systemic signaling mechanisms to coordinate whole-body responses to environmental challenges. Such systemic signaling involves long-distance propagation of electrical, calcium (Ca²⁺), reactive oxygen species (ROS), and glutamate (Glu) signals, which are often interdependent and integrated to orchestrate plant responses in the distal parts as a result of local attacks. Such attacks include numerous biotic or abiotic stresses (e.g., salinity, heat, high light, insect attack, or pathogen infection), which can trigger systemic responses in plants ranging from defense gene expression, metabolic regulation, and even movement of an organ or cells^{1.2}. Furthermore, a plant can also utilize systemic signaling to communicate with nearby plants to build network-acquired acclimation^{3.4}.

Animal central nervous system (CNS) is a highly sophisticated systemic signaling network consisting of specialized neuron cells communicating with one another driven by numerous neurotransmitters and their receptors. Although plants lack the specialized cell types and structures in animals, they appear to retain some of the basic components (e.g., ion channels and second messengers) for long-distance transmission of signals. The plant glutamate receptor-like (GLR) channels are outstanding examples of such systemic signaling components, which represent the homologs of the animal ionotropic glutamate receptors (iGluRs). In Arabidopsis GLR family consists of 20 members of proteins that are classified into three clades (GLR1, GLR2, and GLR3). Concerning their functions in systemic signaling, a well-documented case is the involvement of GLR3.3 and GLR3.6 in the long-distance propagation of electrical, Ca²⁺, and ROS signals elicited by diverse stimuli especially mechanical wounding or insect feeding. The glr3.3glr3.6 double mutant is severely impaired in long distance transmission of wound-induced signals and consequently lacks distal response in jasmonate (JA) accumulation and associated defense against herbivory.

Plant GLR proteins, like animal iGluRs, are ligand-gated Ca²⁺-permeable channels assembled by homo- or hetero-tetrameric subunits. Studies on the GLR3 family members have improved our understanding of the molecular mechanisms underlying GLR channel structure and activation. Increasing evidence supports the hypothesis that GLR3.3 channel is activated by wound-induced diffusion and bulk flow of amino acid agonists in the apoplast, thus triggering long-distance electrical signaling and Ca²⁺ waves⁵. Structural analysis reveals that GLR3.3 has evolved broadly compatible binding sites to accommodate a wide range of amino acid ligands with micromolar affinities (e.g., L-Glu, Gly, L-Met, L-Cys, or ethylene precursor ACC), consistent with observations on GLR3.3-dependent cytosolic Ca²⁺ ([Ca²⁺]_{cyt}) influx in plants⁶. Following activation, many ligand-gated ion channels undergo a desensitized state in which the channel is closed even though the ligand remains bound. As an integral part of channel gating mechanisms, desensitization is necessary for attenuating channel sensitivity to avert overstimulation upon prolonged ligand exposure, or for resuming channel conformation to inactivated state after ligand disassociation. However, little is known regarding the regulatory components and molecular mechanisms underlying plant GLR channel desensitization.

Here we demonstrate the essential role of cytoplasmic C-terminal domain (CTD) in GLR3.3 function. We further identified a mechanism by which the Ca²⁺ sensor calmodulin (CaM) directly binds to a region of CTD to mediate potent desensitization and inactivation of GLR3.3 channel in a Ca²⁺-dependent manner. We found that CRISPR-engineered GLR3.3 mutant allele with impaired Ca²⁺/CaM-mediated desensitization resulted in prolonged wound-triggered long-distance electrical signaling and Ca²⁺ waves. Such alleles rendered plants a more robust systemic JA responses and anti-herbivore defense without impairing plant growth under normal conditions. Our findings have thus revealed a Ca²⁺/CaM-dependent regulatory mechanism for GLR3.3 channel activity through a negative feedback loop and provided a possible breeding target for anti-herbivory traits in crops.

### Results

GLR3.3 C-terminal domain plays a dual role in systemic electrical signaling.

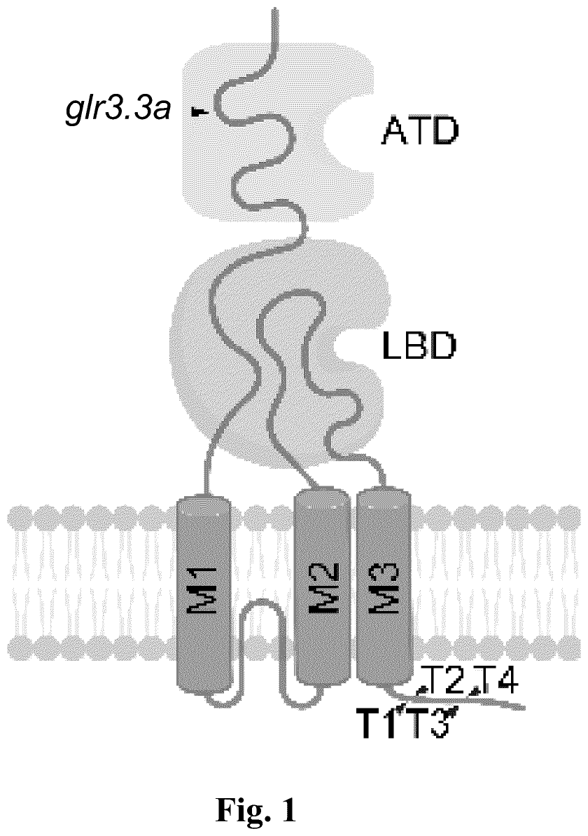

Arabidopsis. GLR channels are homo- or hetero-tetrameric complexes assembled by four subunits, and each subunit consists of a large extracellular amino-terminal domain (ATD), a ligand-binding domain (LBD), a transmembrane domain (TMD) comprising three membrane spanning segments (M1˜M3) plus a cytoplasm-facing reentrant loop, and a short cytoplasmic carboxyl-terminal domain (CTD; FIG. 1a). A recent cryo-electron microscopy (cryo-EM) structure of full-length Arabidopsis GLR3.4 without CTD confirmed the similarity of its overall structure to that of mammalian iGluRs. According to GLR3.4 structure, ATDs and LBDs are responsible for binding to agonist ligands (glutathione and Glu, respectively) which might produce conformational changes for channel opening. The TMDs assemble to form an ion channel pore which contribute to cation permeability and selectivity. However, the cytoplasmic CTD is absent in the structure and its role in GLR channel function remains largely unknown. To characterize the role of CTD in GLR3.3 function, we employed CRISPR (clustered regularly interspaced short palindromic repeats) technique to generate multiple GLR3.3 mutant alleles with varying lengths of CTD (termed 3.3T1, 3.3T2, 3.3T3, and 3.3T4). Among these CRISPR-engineered mutant alleles, 3.3T1 and 3.3T2 lost almost the entire CTD due to premature termination caused by frameshift mutations. The 3.3T3 allele retained most of CTD with only 5 amino acid deletions, and 3.3T4 harbored a truncated CTD due to a premature stop codon (FIG. 1b). In contrast to T-DNA insertional mutant allele glr3.3a, none of these CRISPR-engineered mutant alleles showed obvious difference in GLR3.3 transcript levels, suggesting that mutations in CTD have no effect on GLR3.3 gene expression

To examine whether the mutations in CTD affect GLR3.3 function, we quantitatively compared wound- and Glu-triggered electrical signaling in plants containing the CRISPR mutant alleles using slow wave potential (SWP) recording procedure. In Arabidopsis wild type (WT), wounding primary root of 4-week-old plant rapidly triggered SWP in the leaves, which was characterized by a sustained depolarization phase. Statistical analysis of wound-triggered SWPs showed that the depolarization amplitudes were comparable between all GLR3.3 mutant alleles and WT, but the depolarization durations were significantly reduced in three CRISPR-engineered mutant alleles (3.3T1, 3.3T2, and 3.3T4) resembling that in the T-DNA insertional mutant glr3.3a, suggesting that these CTD mutations represent loss of GLR3.3 function in controlling SWP duration. Intriguingly, unlike the GLR3.3 loss-of-function alleles, 3.3T3 allele showed prolonged durations of wound-triggered SWPs compared to WT, implying that its CTD mutation leads to gain of function in enhancing SWP duration. Glu was recently identified as a major elicitor released into the apoplast upon wounding or herbivore feeding, which is capable of activating GLR channel-mediated long-distance electrical signaling and Ca²⁺ waves. To investigate whether the mutations in CTD affect Glu-triggered electrical signaling, we analyzed the SWPs in systemic leaves of different GLR3.3 mutant alleles after external Glu was applied to wound site of primary root. Statistical analysis of Glu-triggered SWPs showed that the depolarization durations were prolonged in 3.3T3 allele but reduced in other GLR3.3 mutant alleles, similar to the observations of wound-triggered SWPs. Collectively, these results indicate that the CTD is necessary for GLR3.3 function and that different regions of CTD may have different regulatory roles.

To determine whether the altered SWPs are attributable to the specific mutations in GLR3.3 CTD, we introduced a wild type copy of GLR3.3 transgene into the various mutant backgrounds to check if this can restore wild type phenotype. SWP recordings showed that the 3.3T4 loss-of-function phenotype was rescued to wild type levels in 3.3T4 complemented plants. In contrast, 3.3T3 complemented plants still displayed prolonged depolarization durations, reinforcing that 3.3T3 was a dominant gain-of-function allele. Taken together, these results demonstrate that the CTD contains regions for both positive and negative regulations of GLR3.3 function.

A unique region in GLR3.3 binds CaM in a Ca²⁺-dependent manner.

The regulatory domains of ion channels may affect channel activity through physical interactions with the pore-forming domains of the channel, with small ligands, or with other proteins. To understand molecular mechanisms for the intriguing dual roles of CTD in GLR3.3 function, we performed IP-MS (immunoprecipitation and mass spectrometry) to identify intracellular interacting partners of GLR3.3 using transgenic plants expressing GLR3.3-3Flag in the glr3.3/glr3.6 mutant background. Not surprisingly, several GLR family members (GLR2.6, GLR3.2, and GLR3.5) were enriched (Table 1), in agreement with previous findings that GLR members are capable of forming hetero-tetrameric complexes^{7.8}. In addition, two P-type H⁺ ATPases (AHA1 and AHA2) were co-purified with GLR3.3 (Table 1), one of which has been reported to negatively regulate GLR3.3 function.

Multiple calmodulin-type Ca²⁺ sensors (CaMs) were identified as potential partners with GLR3.3 (Table 1), in agreement with a general notion that many Ca²⁺-permeable channels are modulated by Ca²⁺-dependent mechanisms. To confirm the interaction between CaMs and GLR3.3 in planta, we performed co-immunoprecipitation (Co-IP) assay using transgenic plants simultaneously expressing GLR3.3-EGFP and 3Flag-CaM7 proteins. Consistently, CaM7 was co-precipitated with GLR3.3, albeit in absence of external Ca²⁺ addition, and such interaction was slightly increased when 1 mM of external Ca²⁺ was added. As plant cells generally store high concentrations of Ca²⁺ in the apoplast and intracellular compartments, the IP-MS or Co-IP assay in transgenic plants was not the right assay to clarify whether interaction between GLR3.3 and CaMs is dependent on Ca²⁺. To address this question, we attempted to identify the cytoplasmic region of GLR3.3 responsible for interacting with CaM and performed in vitro interaction assay using recombinant proteins. We constructed and expressed in E. coli two fragments of GLR3.3, C1 and C2, that spanned the MI-CTD and M3-CTD, respectively. Compared to the longer CI fragment, the shorter C2 fragment more strongly pulled down CaM7 in the presence of Ca²⁺, demonstrating that the C2 fragment is sufficient to mediate interaction between GLR3.3 and CaM7. Notably, the C2 fragment interacted with CaM2 or CaM7 in the presence of Ca²⁺, and such interaction was completely abolished when Ca²⁺ was absent or chelated by EGTA addition. Moreover, the interaction between C2 fragment and CaM7 occurred in a Ca²⁺-dose dependent manner. Collectively, these findings demonstrated that CaMs directly bind to the CTD of GLR3.3 in a Ca²⁺-dependent manner.

Previous studies have documented that CaM often directly binds to one or more CaM-binding domains (CBDs) of the target proteins to modulate their functions. The canonical CBD is usually characterized by an amphiphilic α-helical structure with a net positive charge in the binding region. Using helical wheel projections, we failed to identify any canonical CBD feature in GLR3.3 CTD, implying that GLR3.3 may contain non-canonical CBD(s). To experimentally identify CBD(s) in GLR3.3 CTD, we constructed a series of CTD deletion variants (designated CD1˜CD4) with in-frame deletions of different regions. Domain mapping with pull-down assays showed that three CTD deletion variants (CD1, CD3, and CD4) strongly interacted with CaM7, similar to the intact CTD (C2), whereas only CD2 failed to interact with CaM7, indicating that the 22-amino acid region deleted from CD2 acts as a putative CBD required for interaction with CaM7. To further clarify whether this CBD is sufficient for interaction with CaM7, the corresponding peptide was chemically synthesized and used to quantitatively measure its binding affinity with CaM7 by isothermal titration calorimetry (ITC) assay. ITC measurements showed that this CBD peptide is sufficient to bind CaM7 with 1:1 stoichiometry in the presence of Ca²⁺ (K_{D})=7.58±1.01 μM), and such binding was severely diminished when Ca²⁺ was chelated by EGTA addition (K_{D})=279.33±20 μM), indicating that only one non-canonical CBD is present in GLR3.3 CTD responsible for binding with CaM.

Ca²⁺/CaM potently desensitizes GLR3.3 channel.

CaM has been connected to the regulation of many ion channels or pumps in plants through distinct regulatory mechanisms. Such regulation can be either activation or inhibition as exemplified by cyclic nucleotide-gated channels (CNGCs) and mildew resistance locus-O (MLO) channels. While, CaM interacts with CNGC18/CNGC8 to open the channel for driving Ca²⁺ oscillations during pollen tube growth, it also interacts with MLO proteins to close the channel for shaping Ca²⁺ oscillations during pollen tube reception. To explore the functional consequences of CaM binding to GLR3.3, we examined the effect of CaM on the Ca²⁺-transporting activity of GLR3.3 channel using single-cell Ca²⁺ imaging assay. After external Glu application, rapid [Ca²⁺]_{cyt} increases were observed in COS-7 cells expressing GLR3.3 alone, while such Glu-activated [Ca²⁺]_{cyt} increases was dramatically inhibited in cells expressing GLR3.3 combined with either CaM2 or CaM7, indicating that CaMs potently desensitize the Glu-induced activity of GLR3.3 channel. Given the Ca²⁺-dependent binding of CaM to GLR3.3 cytoplasmic CTD, we further examined the differential effects of CaM on GLR3.3 channel properties in the presence or absence of internal Ca²⁺ by whole-cell patch-clamp configuration. When free internal Ca²⁺ in pipette solutions was adjusted to 1 μM, large inward currents induced by external Glu application were recorded in HEK293 cells expressing GLR3.3 alone, while such Glu-induced inward currents was severely declined in cells co-expressing GLR3.3 with either CaM2 or CaM7, indicating that CaMs potently desensitize Glu-induced GLR3.3 channel opening in the presence of internal Ca²⁺. In contrast, when free internal Ca²⁺ in pipette solutions was adjusted to 0 nM, the Glu-induced inward currents were not altered, even in cells co-expressing GLR3.3 with CaM2 or CaM7, reinforcing the idea that CaM desensitizes GLR3.3 channel in response to the rise of intracellular Ca²⁺.

CaM specifically binds to a unique CBD in GLR3.3 CTD, implying a key role of this CBD in Ca²⁺/CaM-mediated desensitization of GLR3.3 channel. To investigate whether GLR3.3 channel desensitization results from direct binding of CaM to the CBD of GLR3.3, we compared the Ca²⁺/CaM-mediated desensitization of two GLR3.3 versions (wild type GLR3.3, harboring intact CTD; and GLR3.3-CD2, harboring CBD-deleted CTD). The single-cell Ca²⁺ imaging assays showed that the Glu-activated [Ca²⁺]_{cyt} increases were comparable in COS-7 cells expressing GLR3.3-CD2 and cells expressing GLR3.3, indicating that CBD-deleted GLR3.3 retains Glu-induced channel activity. Interestingly, the Glu-activated [Ca²⁺]_{cyt} increases were not significantly attenuated in COS-7 cells co-expressing GLR3.3-CD2 with CaM7, in contrast to the strong inhibition effect that occurred in cells co-expressing GLR3.3 with CaM7, confirming an essential role of this unique CBD in Ca²⁺/CaM-mediated desensitization of GLR3.3 channel. Consistent with findings in single-cell Ca²⁺ imaging assays, the whole-cell patch-clamp recordings showed that the Glu-induced inward currents in HEK297 cells expressing GLR3.3-CD2 were comparable to those in cells expressing GLR3.3, indicating that CBD is not required for Glu-activated GLR3.3 channel opening. Intriguingly, the Glu-induced inward currents recorded in HEK297 cells co-expressing GLR3.3-CD2 with CaM7 were comparable to those in cells expressing GLR3.3-CD2 alone, regardless of levels in free Ca²⁺ in pipette solutions (0 nM or 1 μM), indicating that GLR3.3 channel desensitization is determined by the CBD-mediated binding of CaM.

Taken together, these results indicate that GLR3.3 channel is desensitized by CaM binding to GLR3.3 in response to elevated Ca²⁺ levels, which may serve as a feedback regulatory loop to orchestrate wound-or Glu-activated signaling outputs.

The CRISPR 3.3T3 mutation impairs Ca²⁺/CaM-mediated desensitization of GLR3.3 channel.

Having shown the essential role of CBD in Ca²⁺/CaM-mediated desensitization of GLR3.3 channel, we noticed that 3.3T3 mutant allele, which caused prolonged wound- and Glu-triggered SWPs in plants, contained a 5-amino acid deletion in the center of CBD, prompting us to delineate the molecular basis for the gain-of-function GLR3.3 channel in 3.3T3 mutant allele. We first evaluated whether the mutated CBD in the 3.3T3 allele affected its binding capacity with CaM7 using pull-down assay. Compared to wild type version (C2), the mutated CBD (designated T3) showed much weaker interaction with CaM7. This was further confirmed by ITC assay in which the mutated CBD peptide was chemically synthesized and used to titrate into CaM7 protein. The CaM binding affinity of the mutated CBD peptide (K_{D})=71.30±14.16 μM) was approximately 10-fold lower than that of wild type CBD (K_{D})=7.58±1.01 μM).

To examine the functional consequences of the mutated CBD, we compared the differential effects of Ca²⁺/CaM-mediated desensitization on two GLR3.3 versions (GLR3.3, harboring wild type CBD; GLR3.3-T3, harboring mutated CBD) using single-cell Ca²⁺ imaging assays and whole-cell patch-clamp recordings. The single-cell Ca²⁺ imaging assays showed that Glu-activated [Ca²⁺]_{cyt} increases in COS-7 cells expressing CBD mutated GLR3.3-T3 exhibited no significant difference with those cells expressing wild type GLR3.3, indicating that the mutated CBD did not affect the Glu-activated GLR3.3 channel opening. Strikingly, in contrast to the obvious inhibition effect of CaM on GLR3.3, the Glu-activated [Ca²⁺]_{cyt} increases in COS-7 cells co-expressing GLR3.3-T3 with CaM7 were comparable to those in cells expressing GLR3.3-T3 alone, indicating that the mutated CBD severely impairs the GLR3.3 channel desensitization mediated by CaM binding. Consistently, the whole-cell patch-clamp recordings showed that Glu-induced inward currents were not significantly affected by the level (0 nM or 1 μM) of free internal Ca²⁺ in HEK297 cells co-expressing GLR3.3-T3 with CaM7, confirming that the mutated CBD severely attenuates Ca²⁺/CaM-mediated desensitization of GLR3.3 channel. Collectively, these data indicate that the mutated CBD in 3.3T3 mutant allele specifically impairs Ca²⁺/CaM-mediated GLR3.3 channel desensitization without affecting Glu-activated channel opening, which may explain the gain-of-function of GLR3.3 channel and prolonged duration of SWPs in plants harboring this mutant allele.

Having shown that the increased intracellular Ca²⁺ is sensed by CaM to mediate desensitization of GLR3.3 channel for [Ca²⁺]_{cyt} decay, we reasoned that it could serve as a feedback regulatory mechanism to orchestrate wound-or Glu-activated signaling outputs. However, such feedback regulatory loop was impaired in 3.3T3 mutant allele due to reduced binding capacity of its cytoplasmic CTD with CaM. To characterize the phenotypic effects of this impaired feedback loop in planta, we quantitatively compared the wound- and Glu-triggered Ca²⁺ waves in different GLR3.3 alleles by introducing a genetically encoded Ca²⁺ indicator GCaMP6s into corresponding mutant backgrounds. Similar to previous observations, wound-triggered [Ca²⁺]_{cyt} wave was rapidly detected at systemic leaves after cutting the primary root of a 4-week-old WT plant, which was characterized by a monophasic waveform with a rapid rise followed by a sustained decay. Comparison of time-course curves and levels of the wound-triggered [Ca²⁺]_{cyt} waves showed that the 3.3T3 plants had [Ca²⁺]_{cyt} peak levels comparable to WT plants, in agreement with that the CBD mutation has no effect on GLR3.3 channel activation. In contrast, [Ca²⁺]_{cyt} levels decayed more slowly in 3.3T3 plants compared to WT plants, indicating that the CBD mutation leads to more sustained systemic Ca²⁺ signaling in 3.3T3 plants upon wounding. Likewise, the Glu-triggered [Ca²⁺]_{cyt} waves were also significantly enhanced in 3.3T3 plants relative to WT plants.

CRISPR-engineered GLR3.3 gain-of-function allele enhances plant systemic wound responses and anti-herbivore defense.

Plants with deficient GLR genes are often susceptible to a wide range of herbivores and pathogens due to compromised defense responses. Consistent with previous observations of GLR3.3 knock-out mutant, most CRISPR-engineered GLR3.3 mutant alleles (3.3T1, 3.3T2, 3.3T4) with CTD mutations showed attenuated wound- and Glu-triggered systemic SWPs and Ca²⁺ waves. Strikingly, a unique GLR3.3 mutant allele (3.3T3) with in-frame mutation in CBD showed increased systemic SWPs and Ca²⁺ waves owing to impaired Ca²⁺/CaM-mediated channel desensitization, prompting us to further characterize the physiological consequences of this GLR3.3 gain-of-function allele in planta.

To assess the effect of 3.3T3 allele on systemic wound responses, we compared the transcript levels of early JA-responsive genes OPR3 and JAZ10 in systemic leaves of different GLR3.3 alleles using quantitative real-time PCR assay (qRT-PCR). After wounding primary roots of 4-week-old plants, the expression levels of OPR3 and JAZ10 were significantly higher in systemic leaves of 3.3T3 plants relative to WT plants. Consistent with the increased expression of JA-responsive genes, the wound-induced accumulation of jasmonic acid (JA) and its bioactive derivative JA-Ile in systemic leaves of 3.3T3 plants were also elevated as compared to WT plants. These data indicated that the GLR3.3 gain-of-function allele leads to enhanced systemic wound responses. To evaluate 3.3T3 allele on plant anti-herbivore defense, new hatched larvae of Spodoptera exigua, a devastating polyphagous pest of numerous field crops, were reared on plants harboring different GLR3.3 alleles. At 9 days after rearing, the larvae gained less weight and inflicted less leaf damage on 3.3T3 plants relative to WT plants, indicating that the GLR3.3 gain-of-function allele leads to enhanced anti-herbivore defense in plants.

Taken together, these findings indicate that engineering GLR3.3 channel via CRISPR-based editing to impair the Ca²⁺/CaM-mediated desensitization could be a potential strategy to enhance plant systemic wound responses and anti-herbivore defense.

Ca²⁺/CaM-mediated desensitization of GLR channels is highly conserved across plant species.

In addition to GLR3.3, other GLR3 members in Arabidopsis, particularly GLR3.1 and GLR3.6, are also engaged in propagating long-distance electrical and Ca²⁺ signals to mount systemic defense responses. To investigate whether Ca²⁺/CaM-mediated desensitization also occurs in other GLR3 members, we compared protein sequences of all GLR3 members and found divergent CTDs among them, that appeared to suggest that Ca²⁺/CaM-mediated desensitization might not be conserved among GLR3 members. To experimentally test this speculation, we examined the binding capacities of CaM to GLR3.1 and GLR3.6, two representative members that have been shown to function redundantly with GLR3.3 in propagating systemic signals. Intriguingly, the pull-down assays showed that the CTDs of GLR3.1 and GLR3.6 strongly interacted with CaM7 exclusively in the presence of Ca²⁺, similar to CTD of GLR3.3, despite divergent CTD sequences. To further assess the functional consequence of CaM binding, we compared the effects of CaM on Glu-activated Ca²⁺-permeable activity of different GLR3 members using single-cell Ca²⁺ imaging assay. Resembling the effect of CaM on GLR3.3, the Glu-activated [Ca²⁺]_{cyt} increases were dramatically reduced in COS-7 cells co-expressing either GLR3.1 or GLR3.6 with CaM7, as compared to cells expressing GLR3.1 or GLR3.6 alone, indicating that the Ca²⁺/CaM-mediated desensitization is highly conserved among different GLR3 members in Arabidopsis.

Having shown that CRISPR-engineered GLR3.3 gain-of-function allele can specifically impair Ca²⁺/CaM-mediated channel desensitization and enhance plant systemic wound signaling and anti-herbivore defense, we reasoned that if this mechanism is conserved in other plant species, it would have implications for crop improvement by engineering similar GLR channels. We analyzed the evolutionary relationship and sequence similarity of Arabidopsis GLR3.3 with its homologs from 11 different crop species. Phylogenetic analysis showed that Arabidopsis GLR3.3 shared closest evolutionary relationship with its homologs in Cruciferae species (e.g., Brassica napus, Brassica oleracea, or Raphanus sativus), and its homologs in dicots seem to be more evolutionarily conserved than those in monocots. Interestingly, the evolutionary relationships between different GLR3.3 homologs are also reflected by the sequence similarities between their CBDs. Sequence alignment showed that GLR3.3 homologs from Cruciferac species Arabidopsis, B. napus, B. oleracea and R. sativus shared a highly conserved CBD located in their respective CTDs, whereas the CBD is extremely divergent between dicot Arabidopsis and monocot Oryza sativa. To further assess whether GLR3.3 homologs in different crop species retain the CaM binding capacity, a close homolog BoGLR3.3 and a distant homolog OsGLR3.1, in relation to Arabidopsis GLR3.3 in the phylogenetic trec, were chosen for experimental analyses. The pull-down assays showed that, as expected, the CTD of BoGLR3.3, like Arabidopsis GLR3.3, interacted with CaM7 in the presence of Ca²⁺. To our surprise, the highly divergent CTD of OsGLR3.1 also interacted strongly with CaM7 in the presence of Ca²⁺. To further address whether the Ca²⁺/CaM-mediated desensitization of GLR channels also occurs in crop species, we examined the effects of CaM on Ca²⁺-permeable activity of different GLR3.3 homologs using single-cell Ca²⁺ imaging assay. After external Glu application, rapid [Ca²⁺]_{cyt} increases were observed in COS-7 cells expressing BoGLR3.3 or OsGLR3.1 alone, indicating their conserved role as Glu-activated Ca²⁺-permeable channels. The Glu-activated [Ca²⁺]_{cyt} increases were dramatically attenuated in cells co-expressing either BoGLR3.3 or OsGLR3.1 with CaM7, as compared to cells expressing BoGLR3.3 or OsGLR3.1 alone, indicating that the Ca²⁺/CaM-mediated desensitization of GLR channels is highly conserved across crop species.

Taken together, these results demonstrate that Ca²⁺/CaM-mediated desensitization of GLR3 channels is a highly conserved regulatory mechanism employed not only by members of Arabidopsis GLR family but also by their homologs in other plant species.

### Methods

### Plant Material and Growth Conditions

Arabidopsis thaliana ecotype Columbia (Col-0) was used as wild type (WT). The glr3.3a (SALK_099757C), glr3.3/glr3.6 (SALK_099757C/SALK_091801C), and GCaMP6s (expressing UBQ10 promoter-driven Ca²⁺-responsive fluorescent protein GCaMP6s) were previously described¹⁵. The pGLR3.3:: GLR3.3-EGFP (expressing GLR3.3 native promoter-driven GLR3.3-EGFP fusion protein) was kindly provided by Dr. Edgar Spalding (University of Wisconsin, Madison) and described previously⁷. The glr3.3a/GCaMP6s was generated by crossing glr3.3a and GCaMP6s and homozygous T3 generation were genotyped by using primers listed in Table 2.

Arabidopsis seeds were surface sterilized and sown on ½ MS agar plates (containing ½ Murashige and Skoog powder, 0.7% agar, and 1% sucrose, pH 5.8). Plates were chilled at 4° C. for 3 days and then transferred to 22° C. growth chamber (with a 16 h light/8 h dark photoperiod) for seed germination. For surface potential recording and whole-plant Ca²⁺ imaging, 1-week-old seedlings were transferred onto new MS agar plates and grown for another 2-3 weeks. For S. exigua feeding assay, 1-week-old seedlings were planted in soil and grown for 4 weeks at 22° C. growth chamber (with a 10 h light/14 h dark photoperiod and 70% humidity).

### CRISPR/Cas9-Based Gene Editing in Arabidopsis

Engineering GLR3.3 C-terminal domain (CTD) was carried out with a YAO promoter-driven CRISPR/Cas9 system as previously described [10]. Briefly, the genomic sequence of GLR3.3 was analyzed with online CRISPR-P 2.0 tool [11], and four specific target regions within GLR3.3 CTD were chosen for designing sgRNAs. The target-specific sgRNAs were synthesized and cloned into pAtU6-26 vector. Then, the complete AtU6-target-sgRNA cassettes were amplified and introduced into Agrobacterium binary vector pCambia1300-pYAO-hSpCas9 for Arabidopsis transformation. The T2 individuals carrying CTD mutations were outcrossed serially to WT for three additional generations to minimize potential off-target effects. The transgene-free progenies with homozygous CTD mutations were verified by PCR and Sanger sequencing. Sequences of sgRNA oligonucleotides and PCR primers are listed in Table 2.

### Genetic Transformation and Crossing

For transgenic plant generation, the full-length coding sequence of GLR3.3 or CaM7 was PCR-amplified from Arabidopsis cDNA and cloned into plant binary vector pCambia1300-3Flag (C terminal on insert) or pCambial300-Flag (N terminal on insert) to generate the p35S:: GLR3.3-3Flag or p35S:: Flag-CaM7 construct, respectively. After verified by sequencing, the constructs were respectively transformed into glr3.3/glr3.6 or Col-0 plants using the Agrobacterium-mediated flower dip method. The homozygous T3 or T4 generation plants of GLR3.3-3Flag/glr3.3/glr3.6 or Flag-CaM7 were employed for further experiments.

For plant crossings, the GLR3.3 mutant alleles (glr3.3a, 3.3T3, and 3.3T4) were crossed with GCaMP6s through pollen hybridizations. The homozygous F3 generation progenies (glr3.3a/GCaMP6s, 3.3T3/GCaMP6s, and 3.3T4/GCaMP6s) were isolated and verified by Sanger sequencing.

PCR primers used for plasmid constructions and Sanger sequencing are listed in Table 2.

### Slow Wave Potential (SWP) Recording

The configuration and procedure for slow wave potential recording were conducted as described previouslyl⁵. Briefly, the 4-week-old plant grown on agar plate was wounded by cutting the primary root at a site 2 cm away from the root-shoot junction. For glutamate treatment, 10 μL of glutamate (100 mM, pH 5.5) was applied to the cutting site after 15-min recovery. To set up a recording configuration, the reference electrode was inserted into the agar medium, and the recording electrode was connected to leaf surface using a drop of 0.5% agar containing 10 mM KCl. The leaf surface potential was recorded by an Axopatch 200B patch-clamp amplifier with a Digidata 1550 digitizer (Axon Instruments). Data acquisition and analysis were performed with pClamp 10.7 software (Axon Instruments).

### Recombinant Protein Preparation

To construct MBP-fusion, GST-fusion, or 6His-fusion plasmids, the coding sequences were amplified from cDNA of Arabidopsis, Brassica oleracea, or Oryza sativa, and respectively inserted into pMal-C5X (MBP-C1, MBP-C2, MBP-T3, MBP-3.1-C, MBP-3.6-C, MBP-Bo3.3-C, and MBP-Os3.1-C), pGEX-6P-1 (GST-CaM2 and GST-CaM7), or pET28a (6His-CaM7). The coding sequences of CD1˜CD4 were synthesized by Huada Biotech (China) and further cloned into pMal-C5X (MBP-CD1˜CD4). Primers for plasmid constructions are listed in Table 2.

Recombinant proteins were produced in Escherichia coli strain BL21 (DE3) and induced with 0.3 mM isopropyl β-D-thiogalactoside (IPTG) for 18 h at 16° C. GST-fusion proteins were purified by Glutathione Sepharose 4B (GE Healthcare) and cluted by 10 mM GSH. MBP-fusion proteins were purified by Amylose Resin (New England Biolabs) and cluted by 20 mM Maltosc. 6His-fusion proteins were purified by NTA Agarose (Qiagen) and eluted by 250 mM Imidazole. All recombinant proteins were further purified by HiTrap Desalting chromatography (GE Healthcare) with a working buffer containing 10 mM HEPES, pH 7.4, 150 mM NaCl, and 1 mM DTT.

### Pull-Down Assay

Approximately 30 μg of purified MBP-fusion proteins were immobilized with Amylose Resin for 2 h at 4° C. in reaction buffer (50 mM Tris-HCl, pH 7.8, 100 mM NaCl, 0.1% Tween 20, 10% glycerol, and 20 mM β-mercaptoethanol). After washing five times, 10 μg of GST-fusion proteins were added and incubated in reaction buffer supplemented with 1 mM Ca²⁺, 2 mM EGTA, or their combinations. After incubating at 4° C. for 1 h, unbound proteins were washed by corresponding reaction buffer. The precipitated GST-fusion proteins were released by boiling SDS sample buffer at 100° C. for 5 min, and detected by immunoblotting using corresponding antibodies. The relative input abundances of the MBP-fusion proteins were indicated by Ponceau S staining.

### Iimmunoprecipitation Followed by Mass Spectrometry (IP-MS) Assay

To perform IP-MS assay, rosette leaves from 4-week-old soil-grown GLR3.3-3Flag/glr3.3/glr3.6 plants were collected and grinded to a fine powder in liquid nitrogen. Total proteins were extracted in reaction buffer containing 50 mM Tris-HCl, pH 7.4, 150 mM NaCl, 0.1% Nonidet P-40, 1% Triton X-100, 20 mM iodoacetamide, 1 mM phenylmethylsulfonyl fluoride (PMSF), 2× Complete protease inhibitor cocktail and 1× PhosSTOP Phosphatase Inhibitor Cocktail (Roche). The GLR3.3-3Flag proteins were captured with anti-Flag magnetic beads (Sigma-Aldrich) and incubated at 4° C. for 4 h. After washing five times, the precipitated proteins were competitively eluted with 3×FLAG peptides (Sigma-Aldrich), concentrated using spin columns (Millipore), and separated by 10% SDS-PAGE gel. After Coomassie Brilliant Blue staining, the gel pieces with protein bands were excised with a clean razor blade, digested with trypsin at 37° C. overnight, and subjected to mass spectrometric analysis as previously described [12].

The mass spectra data were extracted by Proteome Discoverer (Thermo Fisher, version 1.4) and converted to Mascot generic files. Mascot software (Matrix Science, version 2.5.1) was used to search TAIR database (TAIR10, containing 35,639 protein entries). In the Mascot searches, the carbamidomethylation of cysteine was set as a fixed modification and the oxidation of methionine was set as a variable modification. Trypsin was specified as the proteolytic enzyme and two missed cleavage was allowed. Peptide mass tolerance was set at 10 ppm, fragment mass tolerance was set at 0.8 Da, and peptide charges were set at +2, +3, and +4. Mascot results were filtered with the Mascot percolator to improve the accuracy and sensitivity of peptide identification. Protein identifications were accepted if they were matched by at least 4 unique peptides, each peptide showing probability higher than 80% and with a false discovery rate of <1.0% as determined by the Scaffold Local FDR algorithm. This IP-MS experiment was replicated twice independently, and only proteins identified in both independent experiments with >4 unique peptide matches are shown in the Table 1.

### Co-Immunoprecipitation (Co-IP) Assay

10-day-old seedlings of transgenic plants 35S:: Flag-CaM7 or pGLR3.3:: GLR3.3-EGFPx35S:: Flag-CaM7 were collected and homogenized in protein extraction buffer (25 mM Tris-HCl, pH 7.5, 150 mM NaCl, 5% glycerol, 0.05% Nonidet P-40, 1 mM PMSF, and 1×Complete protease inhibitor cocktail). After protein extraction, 50 μL of anti-GFP magnetic agarose (GFP-Trap MA, ChromoTek) was added to 5˜10 mg of total protein extracts (with or without addition of 1 mM Ca²⁺), and the mixture was incubated for 6 h at 4° C. The precipitated proteins were washed at least four times with the protein extraction buffer and then eluted by boiling SDS sample buffer at 100° C. for 5 min. The clear supernatant was separated in 10% SDS-PAGE gel followed by immunoblotting. The anti-Flag (Sigma-Aldrich) and anti-GFP (ChromoTek) antibodies were used for immunoblot analysis according to the manufacturers' protocols. These experiments were repeated three times with similar results.

### ITC (Isothermal Titration Calorimetry) Assay

The CBD peptide (DQQQNHDSSSMRSTRLQRFLSL; SEQ ID NO:3) and mutated CBD peptide (DQQQNHDSTRLQRFLSL; SEQ ID NO:4) were synthesized by GL Biochem Ltd (Shanghai). ITC was carried out at 25° C. on a MicroCal iTC200 instrument (GE Healthcare). 250 μM of peptides were titrated against 20 μM of 6His-CaM7 proteins (purified by size-exclusion chromatography using Superdex 200 Increase 10/300 GL, GE Healthcare) in the reaction buffer (25 mM HEPES, 100 mM NaCl, pH 7.5) with addition of 1 mM Ca²⁺ or 2 mM EGTA. Titrations were performed by an automated sequence of 20 injections spaced at 150-s intervals to ensure complete equilibration, and raw data were analyzed with Origin software (version 7.0) supplied with the instrument. The K_{D} valve represents mean±SD of 3 biological replicates.

### Single-Cell Calcium Imaging in Mammalian Cells

The full-length coding sequences were amplified from cDNA of Arabidopsis (GLR3.1, GLR3.3, and GLR3.6), B. oleracea (BoGLR3.3) or O. sativa (OsGLR3.1). The coding sequences of GLR3.3-CD2 and GLR3.3-T3 were generated through site-directed mutagenesis with GLR3.3 as template. All coding sequences respectively inserted into a dual-promoter vector pBudCE4.1 (Invitrogen) with coding sequence of GCaMP6s for co-expression in HEK297 or COS-7 cells as previously described. Mammalian cells were cultured in DMEM supplemented with 10% FBS in a 5% CO2 incubator at 37° C. under controlled humidity, and transfected using a Lipofectamine 3000 Transfection Reagent kit (Invitrogen).

COS-7 cells expressing GCaMP6s together with various GLR proteins were monitored by a Zeiss AxioObserver ZI inverted microscope (Ivision 4.5 software) with 2-s intervals under a 20× objective as previously described. The standard solution for Ca²⁺ imaging contained 120 mM NaCl, 3 mM KCl, 1 mM MgCl₂, 1.2 mM NaHCO₃, 10 mM glucose, 10 mM HEPES, pH 7.5. At 60 s after initiation of the imaging procedure, the bath was perfused using a peristaltic pump with the standard solution supplemented with 100 mM glutamate to elicit Ca²⁺ entry through active channels.

### Whole-Cell Patch-Clamp Recording

The whole-cell patch-clamp recordings were performed on an Axopatch 200B patch-clamp configuration with a Digidata 1550 digitizer (Axon Instruments) as described previously. Clampex 10.7 and Clampfit 10.7 software (Axon Instruments) were used for data acquisition and analysis.

To record Ca²⁺ currents across plasma membrane of HEK293 cells, the bath solution contained 100 mM Na-glutamate, 40 mM NaCl, 5 mM KCl, 2 mM MgCl₂, 10 mM CaCl₂, 10 mM HEPES, and 10 mM glucose (pH 7.5, adjusted with NaOH). The pipette solution with 0 nM free Ca²⁺ contained 140 mM CsCl, 5 mM EGTA, and 10 mM HEPES (pH 7.5, adjusted with CsOH), while the pipette solution with 1 μM free Ca²⁺ contained 140 mM CsCl, 5 mM EGTA, 4.85 mM CaCl₂) and 10 mM HEPES (pH 7.5, adjusted with CsOH). Free Ca²⁺ was calculated with Webmaxc Standard software (https://somapp.ucdmc.ucdavis.edu/pharmacology/bers/maxchelator/webmaxc/webmaxcS.htm). A ramp voltage protocol of 2-s duration from −180 mV to +20 mV (holding potential 0 mV) was applied 1 min after achieving a whole-cell configuration, and currents were recorded every 20 s, with 5 repeats in total for each cell. The five current traces were used for statistical analysis to produce average current-voltage curves.

### Whole-Plant Calcium Imaging

Transgenic plants glr3.3a/GCaMP6s, 3.3T3/GCaMP6s, and 3.3T4/GCaMP6s were imaged with a Zeiss Lumar v12 epifluorescence stereoscope equipped with a 6× objective lens and Retiga 12-bit camera. The GCaMP6s fluorescent protein was excited using a mercury lamp (Intensilight Hg Illuminator, Zeiss) and a 440/470-nm excitation filter. The fluorescent signal passing through a 535/550-nm filter was acquired every 2 s with the camera using iVision software (BioVision Technologies). Using the ImageJ software, GCaMP6s signals were analyzed overtime at several regions of interest. To calculate the relative change (ΔF) or maximum change (ΔFₘₐₓ) of fluorescence, the equation ΔF/F₀=(F−F₀)/F₀ or ΔFₘₐₓ=(Fₘₐₓ−F₀)/F₀ was used, where F₀ denotes the average baseline fluorescence of the first 10 frames recorded before the wounding or glutamate treatment.

Insect Feeding Assay with Spodoptera exigua

The S. exigua eggs were purchased from Jiyuan Baiyun Industry Co., Ltd. (China), and hatched at 28° C. in a beaker lined with moist filter papers. Freshly hatched larvae were placed on rosette leaves of 4-week-old plants, and 10 larvae were reared on each plant (24 plants per genotype). After 9 days of feeding, the total larval weight were measured and the average individual larval weight were calculated. Representative larvae were recovered from corresponding genotypes and killed by 75% methanol for taking photos. These experiments were independently repeated four times, and each experiment recovered more than 30 larvae from corresponding genotypes.

Quantitative Real-Time PCR (qRT-PCR)

For GLR3.3 transcript analysis, 7-day-old seedlings of different GLR3.3 mutant alleles were harvested for RNA extraction. For JA-responsive gene analysis, the rosette leaves of 4-week-old plants at 1 h after wounding the primary roots were harvested for RNA extraction. Total RNA was extracted with TRIzol (Invitrogen) and 2 μg of RNA was reverse-transcribed into cDNA with a cDNA Synthesis kit (Invitrogen). qRT-PCR was performed on a BioRad CFX96 qPCR instrument using SYBR Green Mix (Invitrogen). Data was normalized to ACTIN8 expression. All experiments were repeated more than three times; Primers used listed in Table 2.

### Quantification of Jasmonates

The extraction and quantification of jasmonates (JA and JA-Ile) for liquid chromatography-tandem mass spectrometry (LC-MS/MS) analysis were described previously. The d5-JA (CDN Isotopes) or d6-JA-Ile (Quality Control Chemicals) was used as the internal standard for JA or JA-Ile, respectively.

### REFERENCES

1. Kurenda, A., Nguyen, C. T., Chetelat, A., Stolz, S. & Farmer, E. E. Insect-damaged Arabidopsis moves like wounded Mimosa pudica. Proc Natl Acad Sci USA 116, 26066-26071 (2019).
2. Hagihara, T., Mano, H., Miura, T., Hasebe, M. & Toyota, M. Calcium-mediated rapid movements defend against herbivorous insects in Mimosa pudica. Nat Commun 13, 6412 (2022).
3. Szechynska-Hebda, M. et al. Aboveground plant-to-plant electrical signaling mediates network acquired acclimation. Plant Cell 34, 3047-3065 (2022).
4. Lei, Y., Xu, Y., Zhang, J., Song, J. & Wu, J. Herbivory-induced systemic signals are likely to be evolutionarily conserved in euphyllophytes. J Exp Bot 72, 7274-7284 (2021).
5. Bellandi, A. et al. Diffusion and bulk flow of amino acids mediate calcium waves in plants. Sci Adv 8, cabo6693 (2022).
6. Qi, Z., Stephens, N. R. & Spalding, E. P. Calcium entry mediated by GLR3.3, an Arabidopsis glutamate receptor with a broad agonist profile. Plant Physiol 142, 963-71 (2006).
7. Vincill, E. D., Clarin, A. E., Molenda, J. N. & Spalding, E. P. Interacting glutamate receptor-like proteins in Phloem regulate lateral root initiation in Arabidopsis. Plant Cell 25, 1304-13 (2013).
8. Price, M. B., Kong, D. & Okumoto, S. Inter-subunit interactions between glutamate-like receptors in Arabidopsis. Plant Signal Behav 8, e27034 (2013).
9. Ataman, Z. A., Gakhar, L., Sorensen, B. R., Hell, J. W. & Shea, M. A. The NMDA receptor NR1 C1 region bound to calmodulin: structural insights into functional differences between homologous domains. Structure 15, 1603-17 (2007).
10. Iacobucci, G. J. & Popescu, G. K. Resident Calmodulin Primes NMDA Receptors for Ca (2+)-Dependent Inactivation. Biophys J 113, 2236-2248 (2017).
11. Oswald, R. E. New Insights into the Mechanism of Ca (2+)-Dependent Inactivation of NMDA Receptors. Biophys J 113, 2131-2132 (2017).
12. Ishchenko, Y., et al., Regulation of the NMDA receptor by its cytoplasmic domains: (How) is the tail wagging the dog? Neuropharmacology 195, 108634 (2021).
13. XiangWei, W., Jiang, Y. & Yuan, H. De Novo Mutations and Rare Variants Occurring in NMDA Receptors. Curr Opin Physiol 2, 27-35 (2018).
14. Liu, S. et al. A Single-Nucleotide Mutation in a GLUTAMATE RECEPTOR-LIKE Gene Confers Resistance to Fusarium Wilt in Gossypium hirsutum. Adv Sci (Weinh) 8, 2002723 (2021).
15. Shao, Q., Gao, Q., Lhamo, D., Zhang, H. & Luan, S. Two glutamate- and pH-regulated Ca (2+) channels are required for systemic wound signaling in Arabidopsis. Sci Signal 13 (2020).
16. Yan, L. et al. High-Efficiency Genome Editing in Arabidopsis Using YAO Promoter-Driven CRISPR/Cas9 System. Mol Plant 8, 1820-3 (2015).
17. Liu, H. et al. CRISPR-P 2.0: An Improved CRISPR-Cas9 Tool for Genome Editing in Plants. Mol Plant 10, 530-532 (2017).
18. Yan, C. et al. Injury Activates Ca (2+)/Calmodulin-Dependent Phosphorylation of JAV1-JAZ8-WRKY51 Complex for Jasmonate Biosynthesis. Mol Cell 70, 136-149 e7 (2018).

TABLE 1
List of proteins co-enriched with GLR3.3 in IP-MS
(immunoprecipitation and mass spectrometry).

| | | Unique | |
|---|---|---|---|
| Accession | Protein | peptide | |
| Number | Name | number | Protein description |
| AT1G42540 | GLR3.3 | 69 | member of putative ligand-gated ion channel |
| | | | subunit family |
| AT4G10320 | N/A | 62 | tRNA synthetase class I (I, L, M and V) family |
| | | | protein |
| AT5G04140 | GLU1 | 57 | glutamate synthase 1 |
| AT5G26570 | PWD | 43 | chloroplastidic phosphoglucan, water dikinase |
| AT4G20850 | TPP2 | 42 | tripeptidyl peptidase II |
| AT2G18960 | AHA1 | 23 | plasma membrane proton ATPase 1 |
| AT1G56070 | LOS1 | 21 | translation elongation factor 2-like protein |
| AT5G06850 | FTIP1 | 21 | multiple C2 domain and transmembrane region |
| | | | protein 1 |
| AT5G50920 | CLPC1 | 21 | Clp protease ATP-binding subunit homologue |
| AT3G44310 | NIT1 | 17 | nitrilase isoenzyme family |
| AT3G60750 | TKL1 | 17 | transketolase involved in carbon fixation |
| AT4G30190 | AHA2 | 17 | plasma membrane proton ATPase 2 |
| AT1G06950 | TIC110 | 16 | translocon at the chloroplast inner envelope |
| AT4G33010 | GLDP1 | 15 | glycine decarboxylase P-protein 1 |
| AT5G02500 | HSP70-1 | 15 | member of heat shock protein 70 family |
| AT5G17920 | ATMS1 | 14 | cobalamin-independent synthase family protein |
| AT2G41220 | GLU2 | 13 | glutamate synthase 2 |
| AT1G29900 | VEN3 | 12 | carbamoyl phosphate synthetase (CPS) large |
| | | | subunit |
| AT2G39730 | RCA | 12 | rubisco activase |
| AT2G42600 | PPC2 | 12 | phosphoenolpyruvate carboxylase protein 2 |
| AT5G02490 | HSP70-2 | 12 | member of heat shock protein 70 family |
| AT5G11180 | GLR2.6 | 12 | member of putative ligand-gated ion channel |
| | | | subunit family |
| AT2G26080 | GLDP2 | 11 | P-subunit of glycine decarboxylase |
| AT3G43810 | CAM7 | 11 | EF hand domain protein encodes a calmodulin |
| AT2G21390 | N/A | 10 | coatomer, alpha subunit |
| AT2G32390 | GLR3.5 | 9 | member of putative ligand-gated ion channel |
| | | | subunit family |
| AT3G45140 | LOX2 | 9 | chloroplast lipoxygenase 2 |
| AT1G69830 | AMY3 | 8 | plastid-localized α-amylase |
| At2g27030 | CAM5 | 7 | EF hand domain protein encodes a calmodulin |
| AT2G30110 | UBA1 | 7 | ubiquitin-activating enzyme (E1) |
| At2g41110 | CAM2 | 7 | EF hand domain protein encodes a calmodulin |
| AT3G53420 | PIP2; 1 | 7 | plasma membrane intrinsic protein 2A |
| At3g56800 | CAM3 | 7 | EF hand domain protein encodes a calmodulin |
| AT4G35100 | PIP2; 7 | 7 | plasma membrane intrinsic protein 3 |
| AT1G20620 | CAT3 | 6 | catalase catalyzes the breakdown of hydrogen |
| | | | peroxide |
| AT4G35090 | CAT2 | 6 | catalase catalyzes the breakdown of hydrogen |
| | | | peroxide |
| AT5G26000 | TGG1 | 6 | member of glycoside hydrolase family 1 |
| AT1G01790 | KEA1 | 5 | K⁺ efflux antiporter 1 |
| AT5G60660 | PIP2; 4 | 5 | plasma membrane intrinsic protein 2; 4 |
| AT1G42970 | GAPB | 4 | glyceraldehyde-3-phosphate dehydrogenase B |
| | | | subunit |
| AT3G48110 | EDD1 | 4 | glycine-tRNA ligase |
| AT4G11420 | EIF3A | 4 | a subunit of eukaryotic initiation factor 3 (eIF3) |
| AT4G35290 | GLR3.2 | 4 | member of putative ligand-gated ion channel |
| | | | subunit family |
| AT5G21274 | CAM6 | 4 | EF hand domain protein encodes a calmodulin |

The IP-MS experiment was replicated twice independently using GLR3.3-3Flag/glr3.3/glr3.6 complementation plants, and only proteins identified in both independent experiments with ≥4 unique peptide matches are shown in the table. N/A denotes no available protein name.

TABLE 2
List of primers
qRT-PCT

| Gene | Forward (5′→3′)/Reverse (5′→3′) Primers |
|---|---|
| JAZ10 | ATCCCGATTTCTCCGGTCCA (SEQ ID NO: 5) |
| | ACTTTCTCCTTGCGATGGGAAGA (SEQ ID NO: 6) |
| OPR3 | AAGATTCGATCTCTCTCATCGAGT (SEQ ID NO: 7) |
| | GGAGTGGTCCGTTGAGCA (SEQ ID NO: 8) |
| GLR3.3 | CGACCTTTCAACCGTCTTAT (SEQ ID NO: 9) |
| | TCGAGAAGCTAAACCAGAGAA (SEQ ID NO: 10) |
| ACTIN8 | TCAGCACTTTCCAGCAGATG (SEQ ID NO: 11) |
| | CTGTGGACAATGCCTGGAC (SEQ ID NO: 12) |

Vector Construction

| Construct | Forward (5′→3′)/Reverse (5′→3′) Primers |
|---|---|
| p35S-GLR3.3-3Flag | gactctagaaagcttctgcaggggcccgggATGAAGCAACTCTGGACTTTCTTCTTC |
| | (SEQ ID NO: 13) |
| | tcatggtctttgtagtccatggtaccGTCTAATGGATTTACCGAATTGAAG |
| | (SEQ ID NO: 14) |
| p35S-Flag-CaM7 | acgcgtcgacATGGCGGATCAGCTAACCGATG |
| | (SEQ ID NO: 15) |
| | ctagactagtTCACTTTGCCATCATGACTTTGAC |
| | (SEQ ID NO: 16) |
| PGEX-CaM2 | ccccaattgATGGCGGATCAGCTCACAGACGATCAGATCTCAG |
| | (SEQ ID NO: 17) |
| | ccgctcgagTCACTTAGCCATCATAACCTTCACAAACTCTTCG |
| | (SEQ ID NO: 18) |
| pGEX-CaM7/pET28a- | ccggaattcATGGCGGATCAGCTAACCGATGACCAGATCTCC |
| CaM7 | (SEQ ID NO: 19) |
| | ccgctcgagTCACTTTGCCATCATGACTTTGACGAACTCTTC |
| | (SEQ ID NO: 20) |
| pMAL-C1 | acgcgtcgacCTTATGTGGGCTGTCACGGGTTGTTG |
| | (SEQ ID NO: 21) |
| | ccaattgTTAATGGTGATGGTGATGATGGTCTAATGGATTTACCG |
| | (SEQ ID NO: 22) |
| pMAL-C2 | acgcgtcgacCTCGCTCTCTTCCTATACTTTGTTC |
| | (SEQ ID NO: 23) |
| | ggaattcTTAATGGTGATGGTGATGATGGTCTAATGGATTTACCG |
| | (SEQ ID NO: 24) |
| pMAL-3.1-C | acgcgtcgacAAAATAATCCGCGACTTCTGC |
| | (SEQ ID NO: 25) |
| | ccggaattcTTAATGGTGATGGTGATGATGTATGGGTCTTCTAG |
| | (SEQ ID NO: 26) |
| pMAL-3.6-C | acgcgtcgacTTGATGATACGTCAGTTTGGACAG |
| | (SEQ ID NO: 27) |
| | ccggaattcTTAATGGTGATGGTGATGATGGTTGCAGCGACTTGA |
| | (SEQ ID NO: 28) |
| pMAL-Bo3.3-C | acgcgtcgacCAGATCATCCGTCAGCTCTACAATG |
| | (SEQ ID NO: 29) |
| | ccggaattcTTAATGGTGATGGTGATGATGATCTAATGGATTAAC |
| | (SEQ ID NO: 30) |
| pMAL-Os3.1-C | acgcgtcgacCACGCCTGCAACCTCTTCTACCAG |
| | (SEQ ID NO: 31) |
| | ccggaattcTTAATGGTGATGGTGATGATGGCATGAAGCGGTGGT |
| | (SEQ ID NO: 32) |
| pBudCE4.1-CMV- | aggctagcctcgaggaattcATGGGTTCTCATCATCATCATC |
| GCaMP6s | (SEQ ID NO: 33) |
| | atgagtttttgttcggatccTCACTTCGCTGTCATCATTTGTAC |
| | (SEQ ID NO: 34) |
| pBudCE4.1-EF-1α- | caggtgtcgtgaacacgtggtcgcggccgcATGTTAAGCTCAATGAATTG |
| GLR3.1 | (SEQ ID NO: 35) |
| | gagatctggcctcgagtccggtaccgaatTCATATGGGTCTTCTAGATGC |
| | (SEQ ID NO: 36) |
| pBudCE4.1-EF-1α- | caggtgtcgtgaacacgtggtcgcggccgcATGAAGCAACTCTGGACTT |
| GLR3.3 | (SEQ ID NO: 37) |
| | gagatctggcctcgagtccggtaccgaatTCAGTCTAATGGATTTACCG |
| | (SEQ ID NO: 38) |
| pBudCE4.1-EF-1α- | caggtgtcgtgaacacgtggtcgcggccgcATGAAGTGGTTTCTGCTT |
| GLR3.6 | (SEQ ID NO: 39) |
| | gagatctggcctegagtccggtaccgaatTTAGTTGCAGCGACTTGAAC |
| | (SEQ ID NO: 40) |
| pBudCE4.1-EF-1α- | caggtgtcgtgaacacgtggtcgcggccgcATGAAGCTACTCTGGTCT |
| BoGLR3.3 | (SEQ ID NO: 41) |
| | gagatctggcctcgagtccggtaccgaatTCAATCTAATGGATTAACCGAAT |
| | (SEQ ID NO: 42) |
| pBudCE4.1-EF-1α- | caggtgtcgtgaacacgtggtcgcggccgcATGAAGTTCATTTTCTATC |
| OsGLR3.1 | (SEQ ID NO: 43) |
| | gagatctggcctcgagtccggtaccgaatTTAGCATGAAGCGGTGGTG |
| | (SEQ ID NO: 44) |
| pBudCE4.1-EF-1α-CaM2 | caggtgtcgtgaacacgtggtcgcggccgcATGGCGGATCAGCTCACAGACG |
| | (SEQ ID NO: 45) |
| | gagatctggcctcgagtccggtaccgaatTCACTTAGCCATCATAACC |
| | (SEQ ID NO: 46) |
| pBudCE4.1-EF-1α-CaM7 | caggtgtcgtgaacacgtggtcgcggccgcATGGCGGATCAGCTAACCGATG |
| | (SEQ ID NO: 47) |
| | gagatctggcctcgagtccggtaccgaatTCACTTTGCCATCATGACTTTG |
| | (SEQ ID NO: 48) |

sgRNA Construction

| Target | Forward (5′→3′)/Reverse (5′→3′) Primers |
|---|---|
| pAtU6-T1-sgRNA | GATTGCTTGCAATGGCATCATCGGT (SEQ ID NO: 49) |
| | AAACACCGATGATGCCATTGCAAGC (SEQ ID NO: 50) |
| pAtU6-T2-sgRNA | GATTGATCTCTTGCAATGGCATCAT (SEQ ID NO: 51) |
| | AAACATGATGCCATTGCAAGAGATC (SEQ ID NO: 52) |
| pAtU6-T3-sgRNA | GATTGTTGCAAACGAGTGGAGCGCA (SEQ ID NO: 53) |
| | AAACTGCGCTCCACTCGTTTGCAAC (SEQ ID NO: 54) |
| pAtU6-T4-sgRNA | GATTGCAAGAATCTTTGCAAACGAG (SEQ ID NO: 55) |
| | AAACCTCGTTTGCAAAGATTCTTGC (SEQ ID NO: 56) |

Site-directed Mutagenesis

| Gene | Forward (5′→3′)/Reverse (5′→3′) Primers |
|---|---|
| GLR3.3-CD2 | TTGCAAGAATGGATGAGAAAGAAGAGTCCAAGCACGAAAGC (SEQ ID NO: 57) |
| | CATCCATTCTTGCAATGGCATCATCGGTTGGCTTTTTATAGAG (SEQ ID NO: 58) |
| GLR3.3-T3 | CATGACTCCACTCGTTTGCAAAGATTCTTGTCTCTCATGGATG (SEQ ID NO: 59) |
| | ACGAGTGGAGTCATGATTCTGCTGTTGATCTCTTGCAATGGC (SEQ ID NO: 60) |

Genotyping

| T-DNA line | Primer (5′→3′) |
|---|---|
| LbB1.3 | ATTTTGCCGATTTCGGAAC (SEQ ID NO: 61) |
| glr3.3a(SALK_099757C)-LP | GATGCTGCATATGGTTGTGTG (SEQ ID NO: 62) |
| glr3.3a(SALK_099757C)-RP | GTTGAACGATAAGCTTGCGAG (SEQ ID NO: 63) |
| glr3.6(SALK_091801C)-LP | TTCGTTCAAAGGTGGCATAAC (SEQ ID NO: 64) |
| glr3.6(SALK_091801C)-RP | CGACTATGAGGAAAGACGCAG (SEQ ID NO: 65) |

TABLE 3
Sequence alignment of CTDs from wild-type (CT) and CRISPR-engineered
GLR3.3 mutant alleles (T1~T4). The amino acids identical to wild-type sequence are
highlighted, and the numbers denote positions of residues relative to the amino-terminal.

| | |
|---|---|
| CT | QIIRQLYKKPTDDAIARDQQQNHDSSSMRSTRLQRFLSLMDE |
| T1 | QIIRQLYKKPLQEINSRIMTLPPCAPLVCKDSCLSWMRKKSP |
| T2 | QIIRQLYKKPTDECHCKRSTAES------------------- |
| T3 | QIIRQLYKKPTDDAIARDQQQNHD-----STRLQRFLSLMDE |
| T4 | QIIRQLYKKPTDDAIARDQQQNHDSSSMRSTRFAKILVSHG- |
| | \| \| \| \| \| |
| | 850 860 870 880 890 |
| CT | KEESKHESKKRKIDGSMNDTSGSTRSRGFDRERSFNSVNPLD |
| T1 | STKARRERSMVQ------------------------------ |
| T2 | ------------------------------------------ |
| T3 | KEESKHESKKRKIDGSMNDTSGSTRSRGFDRERSFNSVNPLD |
| T4 | ------------------------------------------ |
| | \| \| \| \| |
| | 900 910 920 930 |
| CT | (SEQ ID NO: 66) |
| T1 | (SEQ ID NO: 67) |
| T2 | (SEQ ID NO: 68) |
| T3 | (SEQ ID NO: 69) |
| T4 | (SEQ ID NO: 70) |

1. A plant expressing an engineered glutamate receptor-like protein (GLR3.3) that provides a hypersensitive response to wounding, resulting in enhanced resistance to insect feeding, wherein the engineered GLR3.3 comprises a gain-of-function mutation within the cytoplasmic C-terminal domain (CTD) of the calmodulin (CaM)-binding domain (CBD).

2. The plant of claim 1, wherein the plant demonstrates:
attenuated or impaired Ca²⁺/CaM-mediated GLR3.3 channel desensitization compared with wild type GLR3.3, without affecting Glu-activated channel opening; or
increased expression of JA-responsive genes or elevates wound-induced accumulation of jasmonic acid (JA) or its bioactive derivative JA-Ile in systemic leaves of the plant, as compared to wild type plants; or
prolonged or enhanced durations of wound- and/or Glu-triggered slow wave potentials (SWPs) compared to wild type, or increased systemic SWPs and Ca²⁺ waves owing to impaired Ca²⁺/CaM-mediated channel desensitization.

3. The plant of claim 1, wherein the GLR3.3 is from Cruciferae species Arabidopsis, Brassica napus, Brassica oleracea, or Raphanus sativus.

4. The plant of claim 1 wherein the CBD corresponds to residues 867-888 of Arabidopsis wild type GLR3.3.

5. The plant of claim 1, wherein the mutation comprises a deletion of 1, 2, 3, 4 or 5 residues within CaM-binding domain region.

6. The plant of claim 1, wherein the mutation comprises a deletion of 1, 2, 3, 4 or 5 of a target region of amino acid sequence SSSMR (SEQ ID NO:1) or SSSLR (SEQ ID NO:2).

7. The plant of claim 1, wherein the mutation comprises SEQ ID NO:69.

8. The plant of claim 1, wherein the mutation comprises GLR3.3-T3.

9. The plant of claim 1 further comprising making the mutation using CRISPR procedure.

10. A method of enhancing plant systemic wound signaling and anti-herbivore defense, the method comprising making or growing the plant of claim 1.
